(19) United States
(12) Patent Application Publication
(10) Pub. No.: US 2013/0072397 A1
(43) Pub. Date: Mar. 21, 2013

(54) METHODS FOR THE DIAGNOSIS AND PROGNOSIS OF A TUMOR USING BCAT1 PROTEIN

(71) Applicant: DEUTSCHES KREBSFORSCHUNGSZENTRUM, Heidelberg (DE)

(72) Inventors: Bernhard RADLWIMMER, Heidelberg (DE); Sebastian BARBUS, Heidelberg (DE); Martje TONJES, Heidelberg (DE); Grischa TODT, Heidelberg (DE); Peter LICHTER, Gaiberg (DE); Guido REIFENBERGER, Dusseldorf (DE)

(73) Assignee: DEUTSCHES KREBSFORSCHUNGSZENTRUM, Heidelberg (DE)

(21) Appl. No.: 13/676,458

(22) Filed: Nov. 14, 2012

Related U.S. Application Data
(63) Continuation-in-part of application No. PCT/EP2011/002307, filed on May 9, 2011.
(60) Provisional application No. 61/334,812, filed on May 14, 2010.

(51) Int. Cl. G01N 33/574

(52) U.S. Cl. CPC G01N 33/57484
USPC 506/9; 436/501

(57) ABSTRACT

The present invention relates to methods for the diagnosis of a tumor, in particular a brain tumor, and for the estimation of a prognosis for patients afflicted with such tumor based on the determination of expression of BCAT1 in a patient sample.

### RELATED APPLICATIONS AND INCORPORATION BY REFERENCE

This application is a continuation-in-part application of international patent application Serial No. PCT/EP2011/002307 filed 9 May 2011, which published as PCT Publication No. WO 2011/141153 on 17 Nov. 2011, which claims priority to U.S. provisional patent application Ser. No. 61/334,812 filed 14 May 2010.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

The present invention relates to methods for the diagnosis of a tumor, in particular a brain tumor, and for the estimation of a prognosis for patients afflicted with such a tumor based on the determination of the expression of BCAT1 in a patient sample.

### BACKGROUND OF THE INVENTION

Cancer is the second leading cause of death in the United States after cardiovascular disease. One in three Americans will develop cancer in his or her lifetime, and one of every four Americans will die of cancer. Malignant human gliomas account for the largest number of human malignant brain tumors. So far, the treatment of gliomas includes neurosurgical techniques (resection or stereotactic procedures), radiation therapy and chemotherapy. However, despite these therapies gliomas are considered as nearly incurable as they fail to respond to ionising radiation, chemotherapy and surgical resection. In other words, with these therapies only a very limited prolongation of lifespan of patients can be achieved, i.e. despite these therapies, the average life span after diagnosis is merely 12 to 16 months. The knowledge of prognostic factors might be decisive for the selection of the preferable kind of life prolonging therapy.

Citation or identification of any document in this application is not an admission that such document is available as prior art to the present invention.

### SUMMARY OF THE INVENTION

The technical problem underlying the present invention is (a) providing improved diagnostic methods for a tumor and (b) providing an overall survival or progression prognosis for patients having such a tumor, leading to a distinct decision of a physician for a particular kind of treatment.

The solution to said technical problem is achieved by providing the embodiments characterized in the claims.

IDH1 mutations occur in a high frequency in WHO grade II and III diffuse gliomas. 93% of all IDH1 mutations are characterized by an amino acid exchange R132H. It could be demonstrated that patients harbouring IDH1 mutations have a better prognosis compared to patients without an IDH1 mutation. Similarly, patients with IDH2 mutations also have a better prognosis in comparison to patients without an IDH2 mutation. This effect was found to be independent from other established molecular markers like losses on 1p/19q and methylation of the MGMT promoter. This observation shows that the analysis of the IDH1 or IDH2 status is of great interest in the field of neurooncology and is useful as a prognostic or predictive marker. Moreover, the knowledge of the IDH1 or IDH2 status has consequences for decisions of the attending physician regarding the particular kind of treatment of patients with (diffuse) gliomas.

The method of the present invention overcomes the problems associated with the direct determination of the mutation status of an IDH1/2 gene. An immunohistochemical assay for classifying tumors based on differences in the metabolism of branched chain amino acids was developed. In brain tumors the assay distinguishes tumors harboring mutations in either the IDH1 or IDH2 genes or both from tumors with wild type IDH1 and IDH2 genes. That way the IDH1/2 status and activity of branched chain amino acid metabolism may be determined using tissue slides. Accordingly, this approach is a fast and simple diagnostic and prognostic stratification of tumors based on branched chain amino acid metabolism activity, e.g., of the IDH1 and IDH2 mutation status by immunohistochemistry.

Moreover, the current analysis of the mutation status of the IDH1/IDH2 gene requires either DNA-sequence analysis or immunohistochemical analysis using, e.g., an IDH1-R132H antibody. Mutation analysis of the IDH2 gene requires DNA-sequence analysis. There is no immunohistochemical assay available for mutant IDH2 protein. By use of the method of the present invention results may be generated much faster and less expensive than by DNA sequencing. Unlike DNA sequencing technology, immunohistochemical analysis is routinely available in diagnostic laboratories. Due to its high sensitivity, the technique may also be used for the indirect diagnostic assessment of IDH1 or IDH2 mutation in tissue samples with low tumor cell content. Moreover, immunohistochemical analysis using an anti-BCAT1 antibody is more sensitive and more specific than immunohistochemical analysis using, e.g., the IDH1-R132H antibody. Using the assay of the present invention 100% of the tumors with IDH1 and IDH2 mutations and 98% of the tumors bearing IDH1 and IDH2 wild type proteins could be correctly classified. In contrast, the IDH1-R132H antibody detects only the IDH1 protein with the R132H mutation. However, this mutation only occurs in about 93% of the mutant IDH1 proteins. Furthermore, the IDH1-R132H antibody does not detect mutant IDH2 proteins which occur in about 4% tumors with mutant IDH1 and/or IDH2 proteins. Therefore, the IDH1-R132H antibody will correctly classify only 89% of tumors harboring the mutant IDH1 and IDH2 proteins. Thus, the specificity of the immunohistochemical IDH1 and IDH2 status analysis is significantly increased from about 89% in known methods to 100% in the method of the present invention.

However, the method of the present invention is not restricted to diagnosis of tumors characterized by IDH1/2 mutations but generally allows to make a prognosis as regards the progression of a tumor based on the determination of the level of BCAT1. In other words, the assay of the present invention allows the fast and reliable diagnostic and prognostic classification of tumors based on the activity of branched chain amino acid metabolism as reflected by the level or activity of BCAT1.

Thus, the present invention relates to methods for the diagnosis of a tumor, in particular a brain tumor, and for the estimation of a prognosis for patients afflicted with such tumor based on the determination of expression of BCAT1 in a patient sample.

Accordingly, it is an object of the invention to not encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1: BCAT1-protein expression in astrocytic gliomas. Tumor sections on a tissue microarray were stained using the antibody against the BCAT1 protein. Representative examples of BCAT1-positive (A) and BCAT1-negative (B) tumors are shown.

FIG. 2: Kaplan-Meier plot of BCAT1 protein expression and overall survival. The following examples illustrate the invention, but are not to be construed as limitations of the invention.

### DETAILED DESCRIPTION OF THE INVENTION

The present invention provides a diagnostic method for the estimation of a prognosis for patients afflicted with a tumor, which may comprise

(a) obtaining a tumor sample from a patient; and

(b) determining the concentration of the BCAT1 protein in the sample, preferably by use of an anti-BCAT1 antibody; whereby a patient showing an increased concentration of BCAT1 has a worse prognosis compared to a patient showing a normal concentration of BCAT1.

The term “increased concentration” as used in the present invention means that the concentration is increased at least by a factor of 3 as compared to a control sample, e.g. normal brain tissue found at the resection margin adjacent to the tumor.

The term “prognosis” concerns an estimation of the overall survival time in months.

The term “worse prognosis compared to a patient showing a normal concentration of BCAT1” as used herein means that the probability of having a given remaining expectancy of life is substantially decreased.

Determination of the level of the BCAT1 protein by immunological methods using an anti-BCAT1 antibody is preferred. However, the person skilled in the art knows further assays for determining the concentration of the protein, e.g., assays based on the determination of a biological activity of the protein as a branched chain aminotransferase.

Preferably, said tumor is a brain tumor. However, the method of the present invention is also of diagnostic value for patients with acute myeloid leukemia (AML) and all other types of tumors carrying mutations of the IDH1 and/or IDH2 proteins, as well as for tumors with variable activity of branched chain amino acid metabolism.

The term “tumor sample” or “brain tumor sample” as used herein, refers to a sample obtained from a patient. The tumor sample may be obtained from the patient by routine measures known to the person skilled in the art, i.e., biopsy (taken by aspiration or punctuation, excision or by any other surgical method leading to biopsy or resected cellular material). For those areas not easily reached via an open biopsy, a surgeon may, through a small hole made in the skull, use stereotaxic instrumentation to obtain a “closed” biopsy. Stereotaxic instrumentation allows the surgeon to precisely position a biopsy probe in three-dimensional space to allow access almost anywhere in the brain. Therefore, it is possible to obtain tissue for the diagnostic method of the present invention.

The term “brain tumor” is not limited to any stage, grade, histomorphological feature, invasiveness, aggressiveness or malignancy of an affected tissue or cell aggregation. In particular grade I, grade II, grade III or grade IV brain tumors, and all other types of cancers, malignancies and transformations associated with the brain are included. A preferred brain tumor to be diagnosed by the method of the present invention is a glioma. Preferred are anaplastic astrocytomas, anaplastic oligoastrocytomas and anaplastic oligodendrogliomas, in particular fibrillary astrocytoma WHO grade II, oligoastrocytoma WHO grade II, oligodendroglioma grade II, anaplastic astrocytoma WHO grade III, anaplastic oligoastrocytoma WHO grade III, anaplastic oligodendroglioma grade III or glioblastoma.

The term “antibody” as used herein relates to any type of antibody known in the art. An antibody as used herein includes intact immunoglobulin molecules, as well as fragments thereof, such as Fab, F(ab)2, and Fv, which are capable of binding an epitope of BCAT1. Typically, at least 6, 8, 10, or 12 contiguous amino acids are required to form an epitope. However, epitopes which involve non-contiguous amino acids may require more, e.g., at least 15, 25, or 50 amino acids.

An antibody which specifically binds to the BCAT1 protein may be used in immunochemical assays, such as Western blots, ELISAs, radioimmunoassays, immunohistochemical assays, immunoprecipitations, or other immunochemical assays known in the art. Various immunoassays may be used to identify antibodies having the desired specificity. Numerous protocols for competitive binding or immunoradiometric assays are well known in the art. Such immunoassays typically involve the measurement of complex formation between an immunogen and an antibody which specifically binds to the immunogen.

An antibody useful in the diagnostic method of the present invention may be raised according to well established methods, i.e., an BCAT1 polypeptide (Schuldiner 0, Eden A, Ben-Yosef T, Yanuka O, Simchen G, Benvenisty N. Proc Natl Acad Sci U S A. 1996 July 9;93(14):7143-8) may be used to immunize a mammal, such as a mouse, rat, rabbit, guinea pig, monkey, or human, to produce polyclonal antibodies. If desired, the (poly)peptide used as an immunogen may be conjugated to a carrier protein, such as bovine serum albumin, thyroglobulin, and keyhole limpet hemocyanin. Depending on the host species, various adjuvants may be used to increase the immunological response. Such adjuvants include, but are not limited to, Freund's adjuvant, mineral gels (e.g., aluminum hydroxide), and surface active substances (e.g. lysolecithin, pluronic polyols, polyanions, peptides, oil emulsions, keyhole limpet hemocyanin, and dinitrophenol). Among adjuvants used in humans, BCG (bacilli Calmette-Guerin) and Corynebacterium parvum are especially useful.

Monoclonal antibodies which specifically bind to BCAT1 may be prepared using any technique which provides for the production of antibody molecules by continuous cell lines in culture. These techniques include, but are not limited to, the hybridoma technique, the human B cell hybridoma technique, and the EBV hybridoma technique (Kohler et al., Nature 256 (1985), 495-7). Moreover, a monoclonal anti-BCAT1 antibody is commercially available (BD Biosciences, San Jose, Calif., USA).

Techniques described for the production of single chain antibodies may be adapted using methods known in the art to produce single chain antibodies which specifically bind to the BCAT1 protein. Antibodies with related specificity, but of distinct idiotypic composition, may be generated by chain shuffling from random combinatorial immunoglobulin libraries [Burton, PNAS USA 88 (1991), 11120-3). Single-chain antibodies also may be constructed using a DNA amplification method, such as PCR, using hybridoma cDNA as a template [Thirion et al., Eur. J. Cancer Prey. 5 (1996), 507-11). Single-chain antibodies may be mono- or bispecific, and may be bivalent or tetravalent. Construction of tetravalent, bispecific single-chain antibodies is taught, for example, in Coloma & Morrison, Nat. Biotechnol. 15 (1997), 159-63). Construction of bivalent, bispecific single-chain antibodies is taught in Mallender & Voss, J.Biol.Chem. Xno9 (1994), 199-206).

A nucleotide sequence encoding a single-chain antibody may be constructed using manual or automated nucleotide synthesis, cloned into an expression construct using standard recombinant DNA methods, and introduced into a cell to express the coding sequence. Alternatively, single-chain antibodies may be produced directly using, for example, filamentous phage technology (Verhaar et al., Int. J. Cancer 61 (1995), 497-501).

Antibodies useful in a method of the invention may be purified by methods well known in the art. For example, antibodies may be affinity purified by protein-A protein-G column chromatography. The bound antibodies may then be eluted from the column using a buffer with a high salt concentration.

The invention is not limited to a particular immunoassay procedure, and therefore is intended to include both homogeneous and heterogeneous procedures. Exemplary immunoassays which may be conducted according to the invention include fluorescence polarisation immunoassay (FPIA), fluorescence immunoassay (FIA), enzyme immunoassay (EIA), nephelometric inhibition immunoassay (NIA), enzyme linked immunosorbent assay (ELISA), and radioimmunoassay (RIA). An indicator moiety, or label group, may be attached to the subject antibodies and is selected so as to meet the needs of various uses of the method which are often dictated by the availability of assay equipment and compatible immunoassay procedures. General techniques to be used in performing the various immunoassays noted above are known to those of ordinary skill in the art.

The present invention also provides a method for distinguishing between (a) a tumor characterized by an IDH1- and/or IDH2 protein with a mutation and (b) a tumor characterized by a IDH 1- and/or IDH2 protein without a mutation, which may comprise

(a) obtaining a tumor sample from a patient; and

(b) indirectly determining the presence of a mutation by determining the concentration of the BCAT1 protein in the sample, preferably by use of an anti-BCAT1 antibody; whereby a non-increased concentration of BCAT1 is indicative of a tumor characterized by an IDH1- and/or IDH2 protein with a mutation.

In this context, the term “mutation” means any gain of function mutation that alters the enzymatic reaction leading to the generation of 2-hydroxyglutarate as one of the end products. Examples of such mutations comprise amino acid substitutions affecting amino acid residue 132 in the IDH1 protein including but not limited to the substitutions R132H, R132C, R132S, R132G, and R132L and amino acid substitutions affecting amino acid residue 172 in the IDH2 protein including, but not limited to the substitutions R172K, R172M, and R172W.

The term “decreased concentration of BCAT1” includes substantial absence of BCAT1 (as determined by immunoassays, e.g. immunohistochemistry.)

The method of the present invention described in more detail in the examples overcomes the problems discussed above since it allows an indirect, fast, simple and reliable analysis of the IDH1/2 status by immunohistochemistry.

Preferably, the mutation indirectly detected by use of the method of the invention is a mutation within the IDH1 protein leading to better prognosis. Particularly preferred is a mutation at position R132 of the amino acid sequence of IDH1, e.g., R132H, R132C and R132S. Alternatively, the mutation indirectly detected by use of the method of the invention is a mutation within the IDH2 protein leading to better prognosis. Particularly preferred is a mutation at position R172 of the amino acid sequence of IDH2, e.g., R172K.

Finally, the present invention also provides a method of selecting a therapy modality for a patient afflicted with a tumor, in particular a brain tumor, which may comprise

(a) obtaining a (brain) tumor sample from a patient; and

(b) determining the concentration of the BCAT1 protein in the sample, preferably by use of an anti-BCAT1 antibody; whereby the selection of a therapy modality depends on the concentration of BCAT1.

The terms “therapy modality” or “mode of treatment” refer to a timely sequential or simultaneous administration of anti-tumor, and/or immune stimulating, and/or blood cell proliferative agents, and/or radiation therapy, and/or hyperthermia, and/or hypothermia for cancer therapy. The administration of these may be performed in an adjuvant and/or neoadjuvant mode. The composition of such “protocol” may vary in the dose of the single agent, timeframe of application and frequency of administration within a defined therapy window.

Thus, in a preferred embodiment of the method of the present invention the mode of treatment to be chosen acts on cell proliferation, cell survival, cell motility, and/or angiogenesis.

In a more preferred embodiment, the mode of treatment comprises chemotherapy, administration of small molecule inhibitors, antibody based regimen, anti-proliferation regimen, pro-apoptotic regimen, pro-differentiation regimen, radiation and/or surgical therapy.

The determination of the level of BCAT1 has an important influence on the therapeutic procedure. Currently, anaplastic astrocytomas WHO grade III are separated from glioblastomas WHO grade IV by the presence or absence of necrosis or vascular proliferation. However, the results of the NOA-04 study (Randomized phase III trial of sequential radiochemotherapy of anaplastic glioma with procarbazine, lomustine, and vincristine or temozolomide) show that the prognosis of (histologically defined) anaplastic astrocytomas WHO grade III without IDH1 mutations is more or less identical to the prognosis of glioblastomas WHO grade IV.

In the therapy of malignant gliomas different protocols are applied to anaplastic astrocytomas WHO grade III and glioblastomas WHO grade IV. Later tumors are treated much more radical by combined radio- and chemotherapy whereas patients with anaplastic astrocytomas WHO grade III receive such bimodal therapy only if they are younger than 40 years of age. The IDH1 status allows the identification of those anaplastic astrocytomas WHO grade III that have a prognosis similar to glioblastomas WHO grade IV and that should be treated like such highest malignant brain tumors.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1

### Materials and Methods

(A) Patients

Tumor tissues of astrocytic gliomas were selected from the collections at the Department of Neuropathology, Heinrich-Heine-University Dusseldorf, Germany. All tumors were histologically classified according to the criteria of the WHO 2000 classification of tumors of the nervous system, which in case of astrocytic gliomas have been retained in the revised WHO classification of 2007.1. Clinical samples were obtained after informed consent and follow-up data of the patients retrospectively determined and linked to the molecular data in an anonymized manner as approved by the Institutional Review Board of the Medical Faculty, Heinrich-Heine-University, Dusseldorf

Paraffin blocks from some tumors were available for the construction of tissue micro-arrays (TMAs). HE (hematoxylin and eosin) stained sections from the donor blocks were used to define representative tumor regions. Up to three different tissue cylinders with a diameter of 0.8 mm were taken from each donor block from selected areas using a tissue chip microarrayer (Beecher Instruments, Sun Prairie, Wis., USA) and transferred to a recipient paraffin block. The recipient paraffin block was cut in 5 ₗam paraffin sections using a Leica DSC1 microtome (Leica Microsystems, Wetzlar, Germany).

(B) Immunohistochemical Detection of BCAT1 Protein

Tumor tissue sections and tumor tissue microarrays were deparaffinized using xylol and rehydrated with graded ethanol. Antigen retrieval was performed by heating for 40 min in a steamer in 10 mM sodium citrate buffer (pH 6.0). Endogenous peroxidase was inactivated by incubating the tissues in 3% hydrogen peroxide. TMAs were incubated overnight with primary anti-ECA39 (BCAT1) monoclonal antibody, clone 51 (BD Biosciences, San Jose, Calif.) diluted 1:3000 in Dako REAL™ antibody diluent (Dako, Glostrup, Denmark). Staining for detection of bound antibody was performed according to standard protocols using the DakoREAL™ Detection System (Peroxidase/DAB+, rabbit/mouse) (Dako, Glostrup, Denmark), subsequent counterstaining was done using hematoxylin. Tumors were scored as either positive or negative depending on the intensity of BCAT1 staining Evaluation of immunohistochemical staining was carried out blinded from clinical data.

(C) Mutation analyses

The IDH1 and IDH2 genes were investigated for mutations using the primers IDH1-sense 5′-accaaatggcaccatacgaa-3′ and IDH1-antisense 5′-acatgcaaaatcacattattgcc -3′ that amplify a 168-bp IDH1-fragment spanning codon 132 or IDH2-sense 5′-ccaatggaactatccggaac-3′and IDH2-antisense 5′-tgtggccttgtactgcagag-3′amplifying a 227-bp IDH2-fragment including codon 172, respectively. The PCR products were purified and sequenced in both directions using cycle sequencing and an ABI PRISM 377 semi-automated DNA sequencer (Applied Biosystems, Foster City, Calif.).

### Example 2

### Analysis of BCAT1 Protein Expression in Different Tumor Samples by Immunohistochemistry

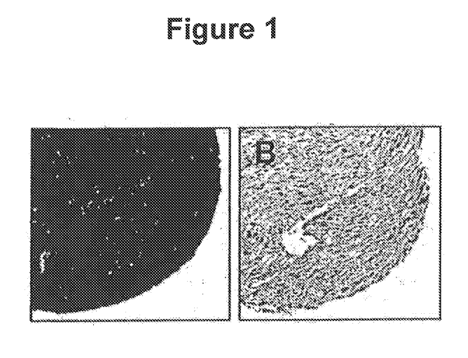

BCAT1 protein expression was analyzed by immunohistochemistry in 81 glioma samples, including 55 glioblastomas of WHO grade IV, 12 anaplastic astrocytomas of WHO grade III (AAIII), 3 anaplastic oligodendrogliomas WHO grade III (AOIII), 10 diffuse astrocytomas of WHO grade II (AII), and 1 oligodendroglioma of WHO grade II. The glioblastoma group included 51 primary glioblastomas (pGBIV) and 4 secondary glioblastomas (sGBIV). 32 of the tumors were stained as whole-tumor sections and 39 as part of a tissue microarray. BCAT1 stainings were scored as either positive or negative. Typical staining patterns representing these two categories are shown in FIG. 1.

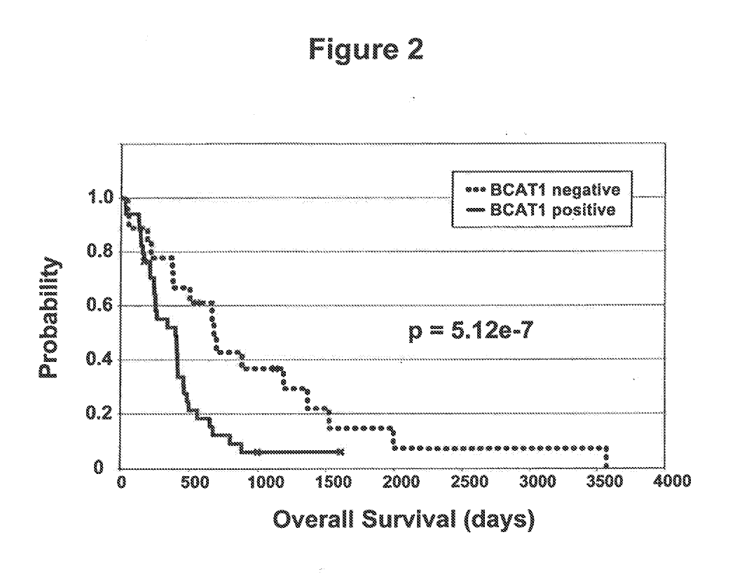

Correlation of BCAT1 protein expression and overall survival was possible for 67 tumor samples for which survival data was available. Of these tumors 43 showed high BCAT1 protein expression and 24 showed low BCAT1 protein expression. Survival analysis by the method of Kaplan and Meier indicated a highly significant (p=5.12e-7) correlation of high BCAT1 protein expression with adverse prognosis (FIG. 2).

BCAT1 protein expression also was correlated with the mutation status of the IDH1 and IDH2 genes. For this purpose the mutation status of the IDH1 and IDH2 genes first was determined by sequencing the region including codon 132 of the IDH1 gene and codon 172 of the IDH2 gene. Mutations of the IDH1 gene were found in 32 tumor samples, including 30 R132H, 1 R132C and 1 R132S mutations. Mutations of the IDH2 gene were present in 3 tumors (all R172K). 45 tumors harboured neither IDH1 nor IDH2 mutations. Analysis of these data using the Fisher's Exact test revealed a highly significant inverse correlation of BCAT1 expression and mutation of the IDH1 or IDH2 genes (p=5.23e-22; Table 1). Of note, the BCAT1 did not label any of the tumors harbouring any type of IDH1 or IDH2 mutant proteins.

TABLE 1
Fisher's Exact Test shows a strong inverse correlation
of BCAT1 protein expression and mutation status
of the IDH1 and IDH2 genes (p = 3.53e−22)

| | BCAT1 | BCAT1 |
|---|---|---|
| | Positive tumors | negative tumors |
| Tumors with IDH1 and IDH2 | 45 | 1 |
| wild type genes | | |
| Tumors with IDH1 or IDH2 | 0 | 35 |
| mutant genes | | |

These data show that BCAT1 protein expression, in addition to being a prognostic marker in gliomas (FIG. 1), also is a diagnostic marker that, due to its high specificity and sensitivity, is excellently suited for classifying tumors with and without mutations of the IDH1 and IDH2 genes. To diagnose these prognostically important tumor groups, an antibody specific for the IDH1-R132H mutant protein currently is used. However, since R132H mutations constitute only 93% of the number of IDH1 mutantions and only 89% of the combined number of IDH1 and IDH2 mutations, this antibody will incorrectly classify a significant proportion of gliomas.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A diagnostic method for the estimation of a prognosis for patients afflicted with a tumor, comprising
(a) obtaining a tumor sample from a patient; and
(b) determining the concentration of the BCAT1 protein in the sample;
whereby a patient showing an increased concentration of BCAT1 has a worse prognosis compared to a patient showing a normal concentration of BCAT1.

2. The method of claim 1, wherein said tumor is a brain tumor.

3. The method claim 2, wherein said brain tumor is a glioma.

4. The method of claim 3, wherein said glioma is glioblastoma grade IV, anaplastic astrocytoma grade III, anaplastic oligodendroglioma grade III, diffuse astrocytoma grade II, or oligodendroglioma grade II.

5. The method of claim 3 wherein the tumor is a low grade astrocytoma or an oligodendroglioma.

6. A method for distinguishing between (a) a tumor characterized by an IDH1- and/or IDH2 protein with a mutation and (b) a tumor characterized by an IDH1- and/or IDH2 protein without a mutation, comprising
(a) obtaining a tumor sample from a patient; and
(b) indirectly determining the presence of a mutation by determining the concentration of the BCAT1 protein in the sample;
whereby a non-increased concentration of BCAT1 is indicative of a tumor characterized by an IDH1- and/or IDH2 protein with a mutation.

7. The method of claim 6, wherein said tumor is a brain tumor.

8. The method claim 7, wherein said brain tumor is a glioma.

9. The method of claim 6, wherein said mutation is a mutation of the IDH1 protein.

10. The method of claim 9, wherein said mutation is at position R132 of the amino acid sequence.

11. The method of claim 10, wherein said mutation is R132H.

12. The method of claim 6, wherein said mutation is a mutation of the IDH2 protein.

13. The method of claim 12, wherein said mutation is at position R172 of the amino acid sequence.

14. The method of claim 13, wherein said mutation is R172K.

15. A method of selecting a therapy modality for a patient afflicted with a tumor, comprising
(a) obtaining a tumor sample from a patient; and
(b) determining the concentration of the BCAT1 protein in the sample;
whereby the selection of a therapy modality depends on the concentration of BCAT1.

16. The method of claim 15, wherein said tumor is a brain tumor.

17. The method claim 16, wherein said brain tumor is a glioma.

18. The method of claim 15, wherein said therapy modality acts on cell proliferation, cell survival cell motility, and/or angioneogenesis.

19. The method of claim 18, wherein said therapy modality comprises chemotherapy, administration of a small molecule inhibitor, antibody based regimen, anti-proliferation regimen, pro-apoptotic regimen, pro-differentiation regimen, radiation and/or surgical therapy.
